(19) United States
(12) Patent Application Publication
(10) Pub. No.: US 2011/0033557 A1
(43) Pub. Date: Feb. 10, 2011

(54) PHARMACEUTICAL COMPOSITION AND DIAGNOSTIC AND THERAPEUTIC USE THEREOF

(76) Inventor: Giuseppe Augusto Scigliano, Milano (IT)

Correspondence Address: LUCAS & MERCANTI, LLP, 475 PARK AVENUE SOUTH, 15TH FLOOR, NEW YORK, NY, 10016, US

(21) Appl. No.: 12/935,790

(22) PCT Filed: Apr. 3, 2009
(86) PCT No.: PCT/IB09/05189

§ 371 Date: Sep. 30, 2010

(30) Foreign Application Priority Data
Apr. 4, 2008 (IT) UD2008A000073

(51) Int. Cl. A61K 33/42; A61K 33/40; A61P 43/00

(52) U.S. Cl. 424/601; 424/616

(57) ABSTRACT

A pharmaceutical composition comprises a diluted water solution containing hydrogen peroxide between about 1% and about 40%, at least a stabilizing compound comprised between about 0% and about 0.2% and at least synergizing compound comprised between about 0% and about 1.5% in weight.

### FIELD OF THE INVENTION

The present invention concerns a pharmaceutical composition for diagnostic use and the therapeutic treatment of pathologies of the mucous membranes and skin, in particular, but not only, pathologies of the oral mucous membrane, for example aphthosis of the oral cavity.

### BACKGROUND OF THE INVENTION

It is known that oral aphthosis is a disease caused by autoimmunity, produced by an immunological “cell” reaction mediated by sensitized T lymphocytes, which react directly with the antigen (immunoreaction). The local liberation of lymphokines produced by the lymphocytes determines a harmful activity against the antigenically altered mucous membrane that leads to the elementary lesion, in the form of a single or multiple surface erosion, with an extremely vivid peripheral erythematous ring that can be observed clinically.

From a histopathological point of view, the aphthous lesion, like a center of inflammation or pathologies of other types, produces a central ischemic necrosis with consequent vasculitis, that is, vascular thrombination caused by endothelial swelling, accompanied by peripheral hyperaemia with increased capillary permeability. This leads to the leakage of red corpuscles and inflammation mediators.

It is known that the diagnosis of oral aphthosis is normally based on exclusively clinical criteria, and here is no laboratory sample or significant diagnostic investigation to show the lesions it causes on the oral mucous membrane. Biopsy is rarely useful, at most for differential diagnosis with other pathologies.

The medical therapy of the aphtha is traditionally symptomatic, promoting healing and reducing symptoms, but without eradicating the pathology.

In less severe cases an aspecific and supporting therapy may be advised, rinsing the mouth with mouthwashes based on clorexidine or FANS, or antiviral methisoprinol, anti-histamine suspensions mixed with anti-ulcer drugs, cyto-protectors, anti-acids, or dabbing with local anesthetics, caustics-escharotics such as zinc chloride, policresulen, acemannan polysaccharide extracted from aloe. In other cases it is possible to use mouthwashes based on tetracycline, but this has unwanted effects connected to prolonged use; or, in more severe cases, topic steroids are used, but these are not without unwanted side effects (candidiasis) either.

The results of these therapies are well known: they give only slight improvements to the symptoms and the course of the illness, without intervening on the causes thereof. However, these therapies are not effective in blocking recurring pathologies.

Purpose of the present invention is therefore to achieve a pharmaceutical composition that is effective and easily applied for the diagnosis and/or therapeutic treatment of pathologies of the mucous membranes, oral or extra-oral, or of the skin, including recurring pathologies.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purpose, a pharmaceutical composition according to the present invention comprises a diluted water solution containing between about 1% and 40% of hydrogen peroxide, preferably between about 2.5% and 36%.

Concentrations of hydrogen peroxide that can advantageously be used are, for example, 3%, 7% or 36% in weight.

According to one embodiment of the present invention, the composition comprises at least a stabilizing compound comprised between about 0% and about 0.2%.

According to another embodiment of the present invention, the composition comprises at least a stabilizing compound comprised between about 0.005% and about 0.2%.

According to a variant, said at least one stabilizing compound makes up between about 0.007% and 0.2% in weight.

Another variant provides that said at least one stabilizing compound is comprised between about 0.01% and 0.15% in weight.

One formulation provides that the at least one stabilizing compound comprises preserving agents.

Moreover, the stabilizing compound can comprise sequestrant agents.

The pharmaceutical composition according to the present invention can comprise, as stabilizing compound, a mix of a sequestrant agent, such as tetrasodium EDTA, and preserving agents, such as esters of p-hydroxybenzoic acids (parabens). Normally, the hydrogen peroxide on the market comprises these stabilizing compounds, EDTA and a mix of parabens.

Alternatively, the stabilizing compound can also be a strong acid like orthophosphoric acid (H3PO4).

According to one formulation of the present invention, the composition comprises at least a synergizing compound comprised between about 0% and about 1.5% in weight.

According to another formulation of the present invention, the composition comprises at least a synergizing compound comprised between about 0.005% and about 1.5% in weight.

According to one formulation, at least one synergizing compound is comprised between about 0.005% and about 0.01% in weight.

Another formulation provides at least one synergizing compound between 0.1% and 1%. Another formulation provides the synergizing compound between about 0.1% and 0.3%.

The synergizing compounds can comprise orthophosphoric acid (H3PO4).

One formulation of the present invention provides orthophosphoric acid as a synergizing compound comprised between about 0.005% and 0.01% in weight.

Alternatively, or additionally, as synergizing compound it is also possible to use sodium fluoride. According to a variant, the sodium fluoride can be comprised between about 0.05% and 1% in weight.

Another variant provides the sodium chloride comprised between about 0.1% and 0.30% in weight.

In another formulation, the composition according to the present invention can comprise methylene blue, with an advantageous function as active principle, according to a variant comprised between about 0.001% and 0.15% in weight.

Methylene blue plays an active role in the composition of the invention, with an oxide-reducing function (redox).

Another advantage of methylene blue is its revealing function which, following the change of color from blue to colorless, can indicate that the composition itself has gone bad.

Furthermore, methylene blue also has an advantageous coloring function of the composition according to the present invention.

According to a variant, the composition according to the present invention can comprise one or more vitamins, for example comprised between about 0% and 2%, advantageously between about 0.005% and 1.5% in weight. According to one fonnulation, a vitamin used in the present invention can be ascorbic acid (vitamin C), or vitamin E, or vitamin F: the vitamins C, E or F have the advantageous function as scavengers.

Furthermore, the composition according to the present invention can also include selenium, which enters to be part of the cycle to produce glutathione which has an oxide-reducing function.

One formulation that can be used of the present pharmaceutical composition, expressed in percentages of weight, provides hydrogen peroxide comprised between about 1% and 40%, methylene blue comprised between about 0.001% and 0.15% and orthophosphoric acid comprised between about 0.005% and 0.2%.

Another formulation that can be used comprises hydrogen peroxide comprised between about 2.5% and 36% in weight, methylene blue comprised between about 0.004% and 0.1% in weight, and orthophosphoric acid comprised between about 0.007% and 0.15% in weight.

In the formulations indicated above it is understood that the water is a component to balance the solution, which functions as a pharmacological vehicle. The solution can be mixed in all proportions with water.

The pharmaceutical composition according to the present invention can also contain one or more pharmaceutically acceptable carriers, excipients, colorants, adjuvants and/or coadjuvants.

According to a characteristic feature of the present invention, said pharmaceutical composition can be used for the diagnosis and/or therapy of pathologies of the mucous membranes and skin.

Here and hereafter in the description, by mucous membrane we mean a membrane that covers the internal face of some organic apparatuses, such as the vesical mucosa, the male and female genital mucosa, nasal, intestinal, the region inside the mouth, including the gums, which is kept continuously wet by the secretion of the muciferous gland or other. It consists of an epithelium, facing toward the free surface, and an underlying layer of connective tissue, possibly containing muscular fibers and lymphocytes.

According to another characteristic feature of the present invention, said pharmaceutical composition can be used in the diagnostic treatment of oral pathologies, such as oral aphthosis, in the occasional sporadic forms, recurrent maior, recurrent minor or herpetiform, Behcet's skin-mucous-ocular syndrome or other pathologies having common histological characteristics, cases of ulcers, vasculitis or micro-hemorrhages with or without pain, such as herpetiform lesions, herpes simplex and herpes zoster, ulcerous-necrotic gingivitis, marginal gingivitis, dental pulpitis, acute and chronic perimplantitis and apical paradontitis, dermatological illnesses where the oral cavity is compromised, of autoimmune genesis, which can also entail ulcerous complications for the fingers, as in scleroderma, and erythematous lesions, as in lupus erythematosus discoid, common bullous pemphigus, lichen ruber planus, lichenoid reactions, herpetic or mycotic forms, precancerous states or evident tumors, caused by immunity deficits or genetic, hypersensitivity, burning mouth syndrome and idiopathic dermatopathies unresponsive to current therapies.

According to another characteristic feature of the present invention, said pharmaceutical composition can be used for the therapeutic treatment of oral pathologies, such as oral aphthosis, in the occasional sporadic forms, recurrent maior, recurrent minor or herpetiform, Behcet's skin-mucous-ocular syndrome or other pathologies having common histological characteristics, cases of ulcers, vasculitis or micro-hemorrhages with or without pain, such as herpetiform lesions, herpes simplex and herpes zoster, ulcerous-necrotic gingivitis, marginal gingivitis, dental pulpitis, acute and chronic perimplantitis and apical paradontitis, dermatological illnesses where the oral cavity is compromised, of autoimmune genesis, which can also entail ulcerous complications for the fingers, as in scleroderma, and erythematous lesions, as in lupus erythematosus discoid, common bullous pemphigus, lichen ruber planus, lichenoid reactions, forms of herpes, mycotic, precancerous states or evident tumors, caused by immunity deficits or genetic, hypersensitivity, burning mouth syndrome and idiopathic dermatopathies unresponsive to current therapies.

The use of the pharmaceutical composition according to the present invention for diagnostic and/or therapeutic treatment is particularly effective when the pharmaceutical composition is applied locally directly on the zone to be diagnosed and/or subjected to therapy, for example by means of a wad of cotton soaked in the composition.

In particular, the present pharmaceutical composition has proven effective for use in the diagnostic of oral aphthosis and other pathologies of the oral or extra-oral mucous membranes, since it allows to show rapidly the lesions caused by said pathologies, rendering them quite visible as blotches of opalescent white color on a pink background (the “white marker effect”).

A similar effectiveness of the pharmaceutical composition has been obtained on lesions of the skin, with encouraging therapeutic results.

The precision of the diagnosis is given by the fact that it is based on the specific nature of the enzymatic catalysis which is involved in the diagnosis and treatment, of which more will be said hereafter.

The use of the pharmaceutical composition according to the present invention in the therapy of pathologies as above also has a surprising result in the prompt remission of symptoms and the maintenance over time of said remission.

The effect of the pharmaceutical composition is due to the scission of the active principles contained in the solution of the composition, due to the complex enzymatic system present in the centers of inflammation. In fact, the Applicant has discovered the presence of said enzymatic system in the centers of inflammation both in lesions where the inflammation is mediated by sensitized T lymphocytes, and also of the classic type due to bacteria.

The leakage of red globules, granulocytes, macrophages and other mediators of inflammation causes a considerable liberation of said complex enzymatic system, in particular anti-oxidant enzymes, present in great concentration in particular in the red globules, neutrophils and bacteria. These enzymes present in the tissue of the center of inflammation are hydroperoxidases, including catalase (CAT) and peroxidase for the most part, oxidase (OX), superoxide dismutase (SOD), glutathione peroxidase (G-px) and glucose-6-phosphate dehydrogenase (G6PD).

The present pharmaceutical composition is unstable in the presence even of minimal traces of certain enzymes, including mainly catalase, present in the lesions, which catalyze and promote the decomposition of the hydrogen peroxide into molecular oxygen and water.

The enzymatic system involved, particularly the hydroperoxidases, has a great affinity for the substrate of the pharmaceutical composition, that is, the hydrogen peroxide. Thanks to the high molecular activity of hydrogen peroxide, that is, the number of molecules transformed in one minute by an enzyme molecule, to the high turnover of the enzymatic system involved and to the high catalytic efficiency (more than 40,000 molecules per second), the opalescence phenomenon can be obtained on average every 15 minutes, repeatable as desired.

This repeatability is important for experimental purposes for the treatment of possible tumoral pathologies of the skin and mucous membranes or other particularly severe pathologies, unresponsive to the therapies in use, which could benefit from the high pharmaco-receptoral reactivity of the invention.

The pharmaceutical composition according to the present invention thus functions as an active compound since it is able to liberate oxygen in the nascent state, which causes the formation of extremely small intra-tissue bubbles that are trapped in the mucosa and sub-mucosa, thus rendering obvious the mucous zones that are compromised, with the coloring described above (phenomenon of opalescence).

The formation of oxygen due to the decomposition of hydrogen peroxide mediated by the enzymes, and the effects of said formation, are proof of the presence, unexpected and never before shown in the prior state of the art or in scientific publications, of said enzymes in the centers of inflammation.

The present invention shows, a few seconds after the topical application, all the aphthous lesions, where they are present, of a size from about 1-2 mm up to about 10 mm, with obvious diagnostic advantages, that is, a visual effect and extremely rapid response.

The present invention shows the area of the lesion, as it is a pharmaceutical composition that is based on the evidence, sensitizing the patient to carry out and continue the treatment because he/she can control the consequent results (patient compliance).

In particular, the present invention allows to show “in toto” the area that is pathologically compromised, and provide the development thereof in an expansive way, since the active front of the peripheral ring of the lesion is more clearly marked in white.

The invention is not limited to showing the ulceration proper, but also the peripheral margin of the lesion which is also affected by the vasculitis, and therefore gives a precise final view of the entity of the mucous membranes involved, with an obvious improvement with respect to the state of the art.

The quicker the diagnostic-therapeutic response, the greater the liberalization of enzymes inside the center of infection: this is a sign that the peak of immune activity is being reached. In such cases, the response is acquired in from 5 to 10 seconds, with the lesion assuming an opalescent snow white color on a pink background.

Generally speaking, the treatment that uses the present invention is not palliative, but curative, since it blocks the relapses that can occur in the same place, functioning as an immuno-modulator, exerting an inhibiting effect on the response of the T lymphocytes against the mucous membranes that is no longer recognized by the latter as antigenically normal, but modified. No new aphthas are observed where they have been previously treated with the invention.

The function of the pharmaceutical composition according to the present invention is supported, as we said, by an enzymatic activity that enters into reaction with the solution applied only against the tissues affected by the pathology, and does not interact with healthy tissues, since the enzymatic compartment is in its natural physiological intra-cellular sector (peroxisomes).

This feature allows to extend the application, not only to oral aphthosis but also to illnesses of immune genesis, such as for example lupus erythematosus discoid, common bollous pemphigus and other dermatopathies unresponsive to current therapies.

The test has been carried out successfully also on lichen ruber planus and in lichenoid reactions, with the therapy leading to a successful cure.

The pharmaceutical composition according to the present invention has also been successfully used in the diagnosis of gingivitis, acute and chronic apical paradontitis and perimplantitis, and therefore it was possible to proceed with a suitable therapy to cure these pathological forms.

The use of the pharmaceutical composition according to the present invention has no collateral effects, no unwanted reaction, no allergic reaction and is tolerated by adult patients and younger patients, including children. The invention has the further advantageous that it allows the patient to “self-diagnose” the presence of aphthosis lesions or small excoriations of traumatic origin that may occur on the mucous membranes of the oral cavity, thus directing in a targeted manner the therapeutic treatment carried out using the invention.

The invention can be used continuously in recurrent aphthosis, since it does not give an collateral effects due to prolonged use, unlike the collateral effects, for example, that may be caused by treatment of recurrent infections by steroids, especially for the throat. The present invention also has a bactericide effect, given by its chemical formulation that prevents bacterial super-infection of the lesions.

Furthermore, the present invention also gives an action that promotes extremely rapid re-epithelization (scarring effect) thanks to its chemical formulation that plays a key role in the normal regulation of the cellular cycle, functioning as a proper growth factor. In fact, the oxygen liberated on a tissue level as described above promotes angiogenesis, which is the basis for the regeneration of tissue and is useful in the repair of compromised skin and mucous membranes.

According to a further feature of the present invention, the pharmaceutical composition can be used in the local analgesic treatment of oral pathologies, such as oral aphthosis, in the occasional sporadic forms, recurrent maior, recurrent minor or herpetiform, very advantageous given the great pain that such pathologies can cause, especially in younger patients.

In fact, after application, the pharmaceutical composition has an analgesic and relieving effect on the pain which can increase until it eliminates the pain in the lesion, extending for 4-5 hours, thus allowing the patient to eat and perform oral hygiene without any problem. Actually, this is a local analgesic effect, since the invention brings absence of pain and not absence of sensation in general (anesthesia), leaving intact almost all the physiological sensations that the patient can perceive.

The effect of local analgesia on the aphthosis lesions is achieved in a few minutes, at most about 15 minutes. This effect is due to the antagonism of the chemical mediators of the inflammation (histamine, bradykinin, lymphokines, prostaglandins E and F (PGE1, PGF1)-(PGE2, PGF2), interleukin 1 (IL 1), chemokines . . . ), important substances in the genesis of pain during inflammation.

The invention thus acts on the cell membranes thanks to its high pharmaco-receptoral affinity, cancelling the inflammatory response mediated by the prostaglandins. The invention prevents the depolarization of the nerve cell and therefore the propagation of the impulse along the neuronal paths, with anti-nociceptive effects on the peripheral neurons.

The analgesic effect is long-lasting, which means that the action of the invention in the aphthosis site extends in an inhibitory manner on the synthesis of the prostaglandins by the prostaglandin endoperoxide synthase enzyme or fatty acid cyclooxygenase 1 (COX-1) and cyclooxygenase 2 (COX-2). On this point, we must point out that the addition of methylene blue to the composition according to the present invention is advantageous because methylene blue, playing the role of active principle due to its redox capacities, modifies the COX-1 and COX-2, with a consequent irreversible inhibition of the cyclooxygenase activity.

Furthermore, the composition according to the present invention inhibits the synthesis of the eicosanoid autocoids carried out by the catalase; the normally low activity of the latter is exalted when the concentration of peroxides and free radicals present in the centers of inflammation increases in the cell. This is in correlation to the presence of large quantities of catalases and hydroperoxidases, discussed above.

Finally, the pharmaceutical composition according to the present invention gives an inhibitory effect on the synthesis of isoprostanes, similar in their effects to prostaglandins, which are normally synthesized by the conversion of arachidonic acid produced by catalyzed oxidation from free radicals.

This inhibitory effect is due to the neutralization of the free radicals induced by the activation of the catalase, superoxide dismutase and glutathione peroxidase.

In conclusion, the pharmaceutical composition according to the present invention acts on pain using the following two mechanisms:
inhibiting the substances that act on the nerve ends generating the pain;
directly influencing the neuronal membranes that biochemically conduct the pain stimulus.

The patient is induced to use the pharmaceutical composition according to the present invention, since it quickly determines an analgesic effect on the aphthous lesion in a few minutes, and the analgesic effect continues for a long time.

The local anesthesia effect can also help to discriminate between a case of pain due to aphthosis to be developed or a pain with a dental origin, such as acute pulpitis. In the first case we will have the opalescent white marking of the zone treated and the disappearance of the pain, whereas in the case of pulpitis we will still have the opalescent effect, but the pain will remain.

The pharmaceutical composition according to the present invention has no unpleasant taste, and hardly any smell.

Fundamentally, the maximum correlation between prescribed dose and effect of the pharmaceutical composition has been obtained thanks to the following strong points:
prescribed dose, which determines a good compliance on the part of the patient;
administered dose, thanks to an extremely rapid absorption by the mucous membranes;
high concentration in situ, connected to the development of good tolerance for the pharmaceutical composition;
high intensity of the analgesic effect, due to high interaction between the pharmaceutical composition and the receptor.

Another advantage of the present invention is that it can easily be used both by professional dental surgeons and also by the patients themselves, to show the mucous lesions of aphthosis, identifying them in a few seconds, so it is possible to understand immediately the extent of the primary center which, in normal conditions, is often difficult to observe. This also applies for aphths that are in the first stages of appearance, which are rapidly detected thanks to the present invention.

The solution is preferably preserved in hermetically sealed containers so as to guarantee that it will not perish, such as a single-dose blister with the upper face made of tin foil, which can be torn open only when it is used, and with a lower face shaped as a hemispherical concave support, made of plastic material, which functions as a single-dose container.

### EXAMPLES

In examples 1, 2 and 3 as shown in the following table, there are three different formulations for the pharmaceutical composition according to the present invention, where the percentages of the various components are expressed in weight, except where otherwise specified. Water is used for balancing in the various examples.

| | | | EXAMPLE 3 |
|---|---|---|---|
| | EXAMPLE 1 | EXAMPLE 2 | (ULTRASPEED |
| | (SOFT TEST) | (NORMOTEST) | TEST) |
| Hydrogen | 3% | 7% | 36% |
| peroxide (H2O2) | | | |
| Methylene blue | 0.001% | 0.01% | 0.15% |
| Orthophosphoric | 0.005% | 0.01% | 0.15% |
| acid (H3PO4) | | | |
| Sodium fluoride | 0.1%0.1% | 0.3%0.5% | 1% |
| 0.05% | | | |

The hydrogen peroxide in example 1 is at 10 volumes, in example 2 at 24 volumes and in example 3 at 130 volumes. The word “volumes” indicates the volume of oxygen that a liter of solution can develop at 0° C. and a pressure of 760 mmHg (1 atm). Therefore, for example, the titer at 3% in weight in example 1 corresponds to about 10 volumes of oxygen, that is 10 liters, developed according to the decomposition reaction of the hydrogen peroxide.

In example 1, the hydrogen peroxide can in any case be made to vary between 2.5% and 3.5% in weight.

In example 2, the hydrogen peroxide can in any case be made to vary between 6.5% and 7.5% in weight.

In example 3, the hydrogen peroxide can in any case be made to vary between 29% and 36% in weight.

### EXAMPLE 4

The formulation of example 2 was effectively applied, by rubbing with a wad of cotton, for example about 1 cm in diameter, to diagnose aphthosis of the oral cavity, maior, minor and herpetiform, and rapidly showed, in about 10-15 seconds, the lesions caused by said pathologies, making them clearly visible as blotches of opalescent snow white on a pink background. This coloring lasts for about 15-30 minutes, and then disappears.

For the therapeutic purposes of said pathologies, the use of the pharmaceutical composition in example 2 consists of painting, with a wad of cotton, applied for the duration of about 10-15 seconds and 3-4 times a day on the lesions.

After application, the analgesic effect described above is given, which extends for 4-5 hours, thus allowing the patient to eat and to perform oral hygiene without problems.

The application of the solution according to the present invention blocks any expansion of the lesion, preventing the ulceration from reaching its final sizes, thus beginning the cure from the first applications.

In particular, for recurrent aphthosis minor, three applications a day for 2-6 days are sufficient, compared with the 4-14 days for known treatments, thus considerably reducing the duration of the lesions and accelerating the healing time compared with other therapeutic systems known in the state of the art. In even more favorable cases, a single application was sufficient, or two-three at most. For the maior form and herpetiform, the duration of the illness passes from more than 30 days in known treatments to about 10-15 days.

### EXAMPLE 5

Apart from applications to aphthosis as in example 4, Applicant has experimented the pharmaceutical composition in example 2 on hundreds of patients for the diagnosis of marginal gingivitis, perimplantitis, dental pulpitis and acute and chronic apical paradontitis, giving certain and unequivocal results on the pathology.

The test is the same as the one for aphthosis, lightly rubbing with a wad of soaked cotton on the apparently healthy and integral mucous membranes in the crevicular zone, that is, the surface area of the gums, just under the clinical crown of the dental element.

In the case of marginal gingivitis and perimplantitis, the soaked wad of cotton is rubbed lightly both vestibularly and lingually; in about 5 seconds we have the opalescent snow white color both on the vestibular and on the lingual side, but high, thus indicating that the surface periodontium is compromised, and is deepening. This alerts the operator, who will begin a therapy suitable to preserve the periodontium, which otherwise would risk, in a few hours, being destroyed by the osteolytic phenomenon, aggravating the periodontal bone architecture.

The test averts, surely and precisely, this fearful pathology for the surface and deep periodontium, right from the very first stages of its onset.

For acute and chronic pulpitis and apical paradontitis, the same applies as above, with the difference that the resultant opalescent snow white color is located farther down, toward the vestibular fornix and hence in the zone of the periapex.

If the result of the test is quick, it means that the pathology is at the height of its activity and therefore in the zone concerned there is a great quantity of enzymes released in the tissue of the mucosa and sub-mucosa, which will lead to obtain a immediate highly dynamic reaction with the solution, and a response that is ready within about 10 seconds.

If the paradontitis is already in regression there is still a response, but slower and less decisive, or moderately dynamic.

The same occurs if the patient has taken drugs before carrying out the test, such as antibiotics or anti-inflammatory drugs.

In such cases the color of the mucous tissue will no longer be opalescent snow white, but ivory white, tending toward dull white, as gradually treatment continues toward a cure, after suitable therapy.

Once the test is finished, the white marker effect recedes spontaneously within 5-20 minutes, leaving no trace of itself as the mucous membrane gradually recovers its usual pink color.

In the case of acute pulpitis and apical paradontitis, where the mucous membrane is closed and integral, unlike in aphthosis, the effectiveness of the pharmaceutical composition is due to the great power of hydrogen peroxide to pass through the cell membranes, reaching the center of inflammation subjected to the vasculitis process, and to enter into contact with the hydroperoxidases released by the cell lysis of the erythrocytes, mostly, and by the granulocytes, macrophages and bacteria.

### EXAMPLE 6

The applications in example 4 and 5 were repeated on young patients and children, using the pharmaceutical composition as in example 1 instead of the pharmaceutical composition as in example 2. This because the mucous membranes of young and very young patients are already receptive to the blander formulation of the pharmaceutical composition as in example 1. In this case too, the application times, both for the diagnostic test and also for the therapy, are identical to those described above.

### EXAMPLE 7

The applications as in example 4 and 5 were repeated using the pharmaceutical composition as in example 3, instead of the pharmaceutical composition as in example 2. Example 3 is particularly indicated to accelerate the response to the utmost, quantifiable as in the range of 3-5 seconds. The application of the pharmaceutical composition as in example 3 is indicated exclusively for professional use, by surgeons and dental surgeons.

It is clear that modifications and/or additions of parts may be made to the pharmaceutical composition and its diagnostic and therapeutic use as described heretofore, without departing from the field and scope of the present invention.

It is also clear that, although the present invention has been described with reference to some examples, a person of skill in the art shall certainly be able to achieve many other equivalent forms of pharmaceutical composition and its diagnostic and therapeutic use, having the characteristics as set forth in the claims and hence all coming within the field of protection defined thereby.

1. Pharmaceutical composition comprising a diluted water solution containing hydrogen peroxide between about 1% and about 40%, at least a stabilizing compound comprised between about 0% and about 0.2% and at least a synergizing compound comprised between about 0% and about 1.5% in weight.

2. Pharmaceutical composition as in claim 1, comprising between about 2.5% and about 36% in weight of hydrogen peroxide.

3. Pharmaceutical composition as in claim 1 comprising 3% in weight of hydrogen peroxide.

4. Pharmaceutical composition as in claim 1 comprising 7% in weight of hydrogen peroxide.

5. Pharmaceutical composition as in claim 1 comprising 36% in weight of hydrogen peroxide.

6. Pharmaceutical composition as in claim 1, wherein said at least one stabilizing compound is comprised between about 0.005% and about 0.2% in weight.

7. Pharmaceutical composition as in claim 6, wherein said at least one stabilizing compound is comprised between about 0.007% and about 0.2% in weight.

8. Pharmaceutical composition as in claim 7, wherein the stabilizing compound is comprised between about 0.01% and about 0.15% in weight.

9. Pharmaceutical composition as in claim 8, wherein the stabilizing compound is provided at 0.02% in weight.

10. Pharmaceutical composition as in claim 8, comprising the stabilizing compound at 0.1% in weight.

11. Pharmaceutical composition as in claim 1, wherein the stabilizing compound comprises preserving agents.

12. Pharmaceutical composition as in claim 1, comprising sequestrant agents.

13. Pharmaceutical composition as in claim 1, wherein said at least one synergizing compound is comprised between about 0.005% and about 1.5% in weight.

14. Pharmaceutical composition as in claim 1, wherein at least one of said one or more synergizing compounds is provided between about 0.005% and 0.01% in weight.

15. Pharmaceutical composition as in claim 13, wherein at least one of said one or more synergizing compounds is provided between about 0.1% and 1% in weight.

16. Pharmaceutical composition as in claim 13, wherein at least one of said one or more synergizing compounds is provided between about 0.1% and 0.3% in weight.

17. Pharmaceutical composition as in claim 1, wherein said one or more synergizing compounds comprise orthophosphoric acid.

18. Pharmaceutical composition as in claim 1, wherein said one or more synergizing compounds comprise sodium fluoride.

19. Pharmaceutical composition as in claim 1 comprising one or more pharmaceutically acceptable carriers, excipients, colorants, adjuvants and/or co-adjuvants.

20. Pharmaceutical composition as in claim 1 comprising methylene blue, EDTA, methyl parahydroxybenzoate, propyl parahydroxybenzoate, sodium fluoride.

21. Pharmaceutical composition as in claim 1 comprising methylene blue.

22. Pharmaceutical composition as in claim 21, wherein the methylene blue is comprised between about 0.001% and 0.15% in weight.

23-28. (canceled)
